(19) United States
(12) Patent Application Publication
(10) Pub. No.: US 2007/0238100 A1
(43) Pub. Date: Oct. 11, 2007

(54) Integrated methodologies for the detection and genotyping of human papillomaviruses

(76) Inventors: Ronald C. McGlennen, Edina, MN (US); David J. Olson, Minnetrista, MN (US); Susie Del Rio LaFreniere, Andover, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/290,854

(22) Filed: Nov. 30, 2005

Related U.S. Application Data
(60) Provisional application No. 60/631,654, filed on Nov. 30, 2004.

(51) Int. Cl. C12Q 1/68; C07H 21/04

(52) U.S. Cl. 435/6; 536/24.32

(57) ABSTRACT

Methods and compositions for detecting, typing or characterizing HPV DNA are disclosed. The methods can include isolation of DNA from a patient and amplifying any L1 region of HPV in the sample. The amplicon from the L1 region may be analyzed to determine at least one of the presence of HPV, the type of the HPV and the risk category of any HPV. The compositions can include one or more primer sets for amplification of the L1 region of HPV.

### RELATED APPLICATIONS

This application claims the benefit of priority from a U.S. Provisional Patent Application having Ser. No. 60/631,654, filed Nov. 30, 2004 the disclosure of which is hereby incorporated by reference in its entirety.

### BACKGROUND OF THE INVENTION

1. Field of Invention

The present invention relates to diagnostic assays and, more particularly, to the detection, characterization, and typing of Human Papilloma Virus.

2. Description of the Related Art

Human Papillomavirus (HPV) infections are ubiquitous in nature, and infections of the anogenital tract are extremely common. HPV viruses were first characterized in the 1970's, where, with the use of rudimentary molecular genetic techniques, their nature of circular deoxyribonucleic acid (DNA) epitheliotropic viruses proved them also to be genetically diverse. Subsequently, HPV are known to be part of the Papovaviridae family of protists, which also include the SV-40 and BK viruses, each known to be tumorigenic in their respective animal hosts. HPV have evolved diversely, where today there are known to be more than 120 different viral types that cause a wide spectrum of clinical disease.

HPV types are highly specific to the anatomic site where infections occur. For example, HPV types 1, 2, 5, and 8 are specific to the skin. More commonly, HPV infection is associated with lesions of the genital tract. In males, this includes lesions known as condyloma affecting the genital skin and the penis. In females, a wide variety of HPV types are known to infect the genital skin, the external genitalia, the vulva, vagina and the cervix. From these observations, a notion has emerged that certain HPV types infecting the anogenital tract cause condylomas or genital warts and are uncommonly are associated with malignant lesions. By contrast, another category of HPV types are more often linked with squamous cell carcinomas and adenocarcinomas of the cervix, vagina, and rectum. In the case of an HPV infection of the uterine cervix, selected HPV types are associated with more than 95% of squamous cell carcinomas. Squamous cell carcinomas are the third leading cause of cancer in females in North America.

With the correlation of HPV to certain cancers, there has been an increased emphasis on clinical testing for HPV. The detection and typing of HPV is becoming the standard of patient care for the prevention of cancers of the anogenital tract, and in select examples evaluation of tumors of the skin. The increased testing for HPV has occurred consequent to the effort to better assess the risk of cancer for an individual infected with HPV. Recently, clinical testing for HPV has focused on molecular genetic methods. Recent methods typically characterize viral DNA derived from tissue samples using various types of probes.

A variety of genetic methods have been developed to test for HPV. Three particular technical approaches are commonly used. The most common approach is the use of solution hybridization. In solution hybridization, a mixture of oligonucleotide probes is combined with genomic and viral DNA in solution. These oligonucleotide probes are typically comprised of a series of modified RNA species. If the sample contains DNA in sufficient amounts of one of the HPV type for which there is also a probe, a complex of the probe and DNA may form. In one embodiment of the solution hybridization assay, that DNA/probe complex is secondarily captured and then reacted with some set of chemicals to produce a reportable signal. Frequently, the detection methodologies utilize various signals including fluorescence, chemiluminescence or radioisotopic signals. The sensitivity and specificity of this assay strategy is defined by the design of the oligonucleotide probes and the conditions for the hybridization reactions. The popular commercial assay sold under the trade name Hybrid Capture II by Digene Corporation (Gaithersburg, Md.) is based on this format. In its current configuration, the Hybrid Capture II assay probes for 13 specific HPV types known to confer a high risk of association with malignant tumors of the female genital tract. The performance of this assay in research and clinical applications is well understood. However, recent studies point to the consistent problem of false positive, false negatives and lower sensitivity when this commercial assay is used for routine HPV testing on samples representative of the HPV infections of the genital tract in the community.

A second approach used involves the hybridization of single or multiplexed oligonucleotide probes to their cognate HPV DNA sequences in the context of the intact cells. This technology, known as in situ hybridization, is based on principles similar to other hybridization methods. In situ hybridization can be used to produce a reporter signal that makes visual the nuclei of cells infected with specific HPV types. The potential advantage of this approach is the correlation of cellular changes characteristic of those with pre cancer or cancer and the associated HVP infection. The challenges to this technology are typically, lower sensitivity to viral genome copy number as well as to the variety of HPV types since typically only single probes are used in each hybridization reaction.

The first description to use the polymerase chain reaction (PCR) to detect specific HPV types was by Shibata et. al., who disclosed the application to identify the specific high risk HPV types 16 and 18 (Shibata, Fu et al.; Shibata, Arnheim et al.). Corresponding to this are the disclosures U.S. Pat. Nos. 4,683,195 and 4,683,202 describing the use of PCR to detect other types of nucleic acid in clinical samples. Related disclosures describe assays to detect a wide variety of viral infections including the DNA sequences from samples infected with the human immunodeficiency virus (HIV), HTLV II and HTLV III (European Patent Publication Nos. 229,701 and 269,445).

The most sensitive and selective technology for HPV detection and typing involves the use of consensus oligonucleotide primers for HPV. This strategy combines the quantitative sensitivity of PCR to amplify even minute amounts of the DNA sequence of interest with the qualitative advantage to amplify and identify a broad spectrum of HPV types from a single reaction. As discussed by Manos et. al., the use of a mixture of distinct and separate oligonucleotide primers for HPV detection was described in abstracts from the 7^{th} International Papillomavirus Workshop Campione-Piccardo et. al., May 1988 p 19. Others described a variety of other type specific primers to detect HPV by PCR from that same meeting. The initial disclosure of the use of consensus oligonucleotide primers to detect and type HPV DNA sequences was by Manos et. al in patent application Ser. No. 243,486, filed Sep. 9, 1988. Several improvements and additional disclosures were added to subsequent applications, which was issued as U.S. Pat. No. 5,182,377. In these disclosures, a series of consensus oligonucleotides, disclosed methods that use PCR to amplify selected regions of the HPV genome, followed by the solution hybridization and/or solid phase hybridization with type specific oligonucleotide probes. The design of the preferred oligonucleotide primers described in that invention, known as MY09 and MY11, incorporate one or multiple nucleotides at selected positions within the length of those primer sequences. Those sites within the oligonucleotide created to position multiple nucleotides are known as degenerate sites, and confer to the resulting mixture of primers, select degrees of nonspecificity that permit that primer to anneal to more than one of the potentially large number of HPV encountered in sample of DNA being tested. This strategy has been used successfully in a number of research and clinical based studies demonstrating the capacity of that assay design to detect a majority of HPV infections.

Based on the use of consensus PCR, several other primer systems have been described that variably improve the specificity or sensitivity of assays to detect HPV. A variation of the earlier described MY09/MY11 primers, known as the PGMYO/11 Line Blot Assay primers, are positioned within the L1 region of the HPV genome (Coutlee, Gravitt et al.; Gravitt, Peyton et al.). These primers are designed to work well with oligonucleotide probes, and have been commercialized as a line probe assay (van Doom, Quint et al.). The prescribed advantage of this design is reported to be improved sensitivity of that assay due to the increased PCR efficiency achieved when producing a smaller sized DNA amplicon, however, no specific study has investigated the improvement in specificity due to the altered positioning of the consensus sequence within the L1 region of the HPV genome. The notion that smaller amplicon size improves detection sensitivity has been reported and is exemplified in the design of the SPF₁₀ LiPA (Line Probe Assay) system (Quint, Scholte et al.).

Another version of the HPV detection assay involves the primers known as GP5/GP6 and a variant GP5+/GP6+(de Roda Husman, Walboomers et al.; van den Brule, Pol et al.). These primers are directed within the L1 regions, but like the PGMY primers are internal to the region covered by the MY09/MY11 primers. Several reports compare the relative sensitivity and specificity of the GP series of primers with varied conclusions (Kado, Kawamata et al.). Overall, the GP and MY series primers are described as performing with similar analytic character when applied to samples collected from the genital tract.

### SUMMARY OF THE INVENTION

The present invention provides the techniques and methods for the characterization of human papillomavirus infections from a host of sample types. The basic methods describe procedures to obtain high molecular weight genomic DNA from sources of fresh, fixed and paraffin embedded samples of cells harboring the virus. Most often, the relevant samples are derived from cells from the uterine cervix, similar to those methods to obtain a Pap smear. In certain aspects, the invention may provide for novel uses of the polymerase chain reaction, a particular type of gene chemistry, to amplify a segment of HPV DNA that contains sequence variations that permit the genotyping of each or multiple HPV types. In other aspect, the invention provides for integration of the various operations involved in the clinical testing for HPV as part of a strategy to diagnose and prevent the development of cancers associated with these infections.

In one aspect, the present inventions may include novel oligonucleotide DNA sequences for improved detection and typing of human papillomavirus DNA. The novel oligonucleotide DNA sequences can provide higher sensitivity to detect a broad class of known and unknown HPV types. The novel oligonucleotide DNA sequences may include particular nucleotides at positions within the sequence. The novel oligonucleotide DNA sequences may function as primers that have an improved alignment over designated binding sites for all known HPV types. These novel primers may be designed to work within the context of a complete HPV assay, including a modification of methods to extract genomic DNA from a broad base of sample types, and the methods to analyze the result of HPV tests via a telemedicine web portal.

The present inventions may provide methods for extracting DNA from a biological sample, detecting HPV in the sample, and typing the HPV, if present. The present invention may include:

(a) A novel method of obtaining high molecular weight genomic DNA from sources of fresh, fixed and paraffin embedded samples of cells harboring HPV.

(b) Amplification of HPV sequences using novel oligonucleotide primers, which improve detection and typing of HPV DNA. The primers amplify a broad class of known and unknown HPV types because of the selective inclusion of nucleotides at positions within the primer that allow for a higher degree of binding to different HPV sequences.

(c) Detection of HPV DNA using gel electrophoresis or other methods of separating DNA fragments.

(d) Analysis of HPV DNA to determine the genotype of the HPV in the clinical sample

In one aspect, a molecular diagnostic assay in accordance with the present invention may use a polymerase chain reaction to detect and genotype human papillomaviruses involving a series of forward and reverse oligonucleotide DNA primers that demonstrate complete sequence complementarity (100%) for X viral types (82 HPV viral types), and 95% complementarity for Y viral types (106 HPV viral types).

The oligonucleotide primers may be completely degenerate, i.e. use each of the 4 natural nucleotides in viral DNA at selected positions within the consensus sequence of the L1 open reading frame of HPV. The oligonucleotide primers may show superior sensitivity for detecting a broader number of known and unknown HPV types, subtypes and variants consequent to the design of those primers. The oligonucleotide primers may show superior specificity in a standard PCR assay, and produce fewer nonspecific (HPV negative) DNA amplicons. The oligonucleotide primers may detect HPV when coamplified in the presence of multiplexed molecular diagnostic assay. The oligonucleotide primers may detect HPV when specifically coamplified in the presence of oligonucleotides primers for Chlamydia trachomatis, Neisseria gonorrehae, Herpes simplex types 2, Trichomonas vaginalis and other sexual transmitted organisms. The oligonucleotide primers may be employed in a commercial/clinical laboratory assay for HPV detection and typing. The oligonucleotide primers may be constructed from conventional or naturally occurring nucleotide(s). The oligonucleotide primers may be constructed from synthetic or nucleotide analogs. The oligonucleotide primers may be constructed from a combination of conventional (natural) and synthetic nucleotides or nucleotide analogs.

In another aspect, a molecular diagnostic assay using the oligonucleotide primers may use methods to derive purified nucleic acid from a variety of fresh or fixed tissue sources to serve as the template for detection and genotyping of human papillomavirus. The molecular diagnostic assay may assess the quantity of extraction lysis reagent based on a visual assessment of the cellular pellet volume or size using a sample assessment guide that serves to estimate reagent volume and to distinguish cells from noncellular material. The molecular diagnostic assay may assess the reagent volume based on a physical measure of cell density such as turbidity or other light scatter measures. The molecular diagnostic assay may assess a sample based on the release of a predictable quantity of cells from a capture device such as a resin matrix, treated paper or membrane. The molecular diagnostic assay may assess sample size using a template guide where the necessary reagent volumes are calculated on a web based tool that considers the reactant volume and quantities in the subsequent steps of the assay.

In another aspect, the oligonucleotide primers for HPV used in the molecular diagnostic assay may demonstrate a greater sensitivity to low or small quantities of viral genomes in the sample as compared to previously described PCR based HPV assays.

In another aspect, the molecular diagnostic assay may use oligonucleotide primers for HPV that demonstrate broader specificity for the family of HPV types, that include viral types newly described as well as variants of known types. The molecular diagnostic assay may include degeneracy of DNA sequence at a first position A that increases the complementarity to detect HPV types A, B, C. The molecular diagnostic assay may include degeneracy of DNA sequence at a second position B that increases the complementarity of the assay to detect HPV types D, E, F etc. The molecular diagnostic assay may use oligonucleotide primers for HPV which demonstrate fewer nonspecific side reactions when displayed on electrophoresis systems. The molecular diagnostic assay may use oligonucleotide primers where the complementarity of the 3′ primer binding sequence and the subsequent Z bases are 100% sequence aligned in Z HPV types, and 95% sequence aligned in AA HPV types. The molecular diagnostic assay may use an oligonucleotide primer with complete degeneracy for HPV that incorporates synthetic nucleotides, peptide nucleotides and chemically modified nucleotides at key positions within the primer to improve the primer binding efficiency and avidity in concert with the sequence alignment.

In another aspect, the molecular diagnostic assay may analyze the amplicon derived from PCR using: agarose gel electrophoresis; polyacrylamide gel electrophoresis; capillary electrophoresis; hybridization of the amplicon or an anchor sequence modified amplicon to a DNA microarray or other solid support detection device; and/or electronic detection of HPV amplicons. The electronic detection may measure the change of impedance, the change in current or electrical resistance consequent to the presence or absence of the HPV amplicon as compared to a positive and negative control due to the binding or physical effect of the amplicon in solution or on a surface.

In still another aspect, the molecular diagnostic assay may be directed through a software system where the workflow of each operational step, and the management of sample organization and processing, as well as the result interpretation, are controlled by the software system. A software system and method in which the present inventions may be incorporated is disclosed in U.S. patent application Ser. No. 10/409,337 entitled Genetic Test Apparatus and Method the disclosure of which is incorporated by reference herein in its entirety. In this aspect, the molecular diagnostic assay may include requisition and sample accessioning at the site of testing is controlled through an internet based order test order entry software. Further, the molecular diagnostic assay may include collecting information at the patient interface and automatically uploading it into the laboratory accessioning log. Further, the molecular diagnostic assay may include automatically producing a worklist, batch report and individual patient reports for a batch of tests to be processed and run through an internet based transmission. In addition, the molecular diagnostic assay may include the production of a test report which is automatically placed at a site of a patient's interface, including printed and electronic transmission.

### BRIEF DESCRIPTION OF THE DRAWINGS

FIG. 1 shows the HPV L1 region, and positions of primer pairs used to amplify HPV DNA in this region; and

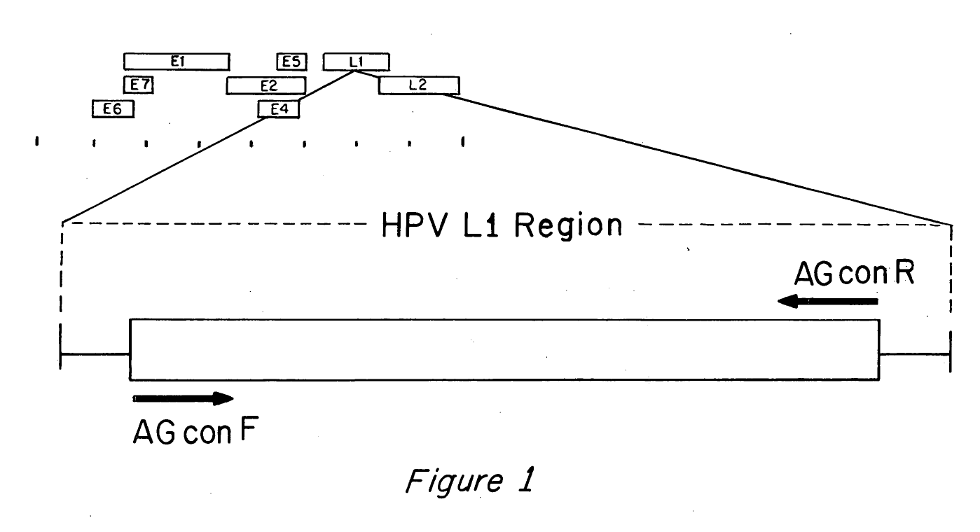

FIG. 2 lists the sequences of the AGconF and AGconR primers and the corresponding HPV types.

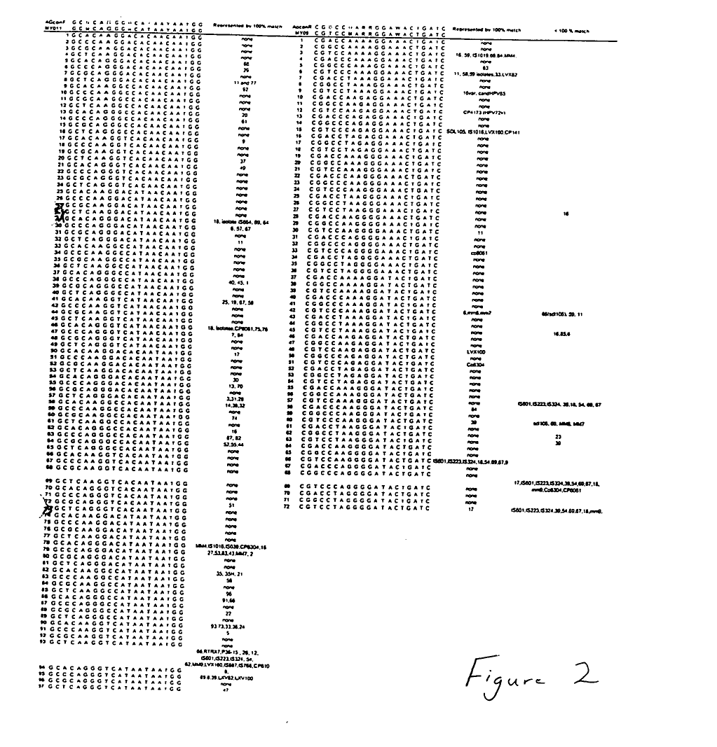

### DETAILED DESCRIPTION OF THE INVENTION

The present invention provides for improved HPV detection by methods of PCR, and details an overall testing procedure that may be cost effective and accurate enough for routine patient diagnostics. The present inventions provide novel methods and compositions for extraction of DNA, oligonucleotide primers, analysis of DNA products from the PCR reactions, interpretation of the results, and the workflow of the entire procedure and the management of the quality assurance and quality control of the testing.

A method in accordance with the present invention may include extracting high molecular weight genomic DNA from clinical samples. The DNA may be extracted from fresh, fixed or paraffin embedded cells. The DNA may be extracted from clinical samples using a number of different methods as will be recognized by those skilled in the art, including, but not limited to, a simple disruption of the cells in the collection solution.

In one method, the cell sample is centrifuged, cells resuspended in a lysis buffer containing a protease. The lysis buffer may include a buffer system such as, for example, TRIS, phosphate, HEPES or other suitable lysis buffer. The lysis buffer may also contain an agent to lyse the cells, which includes, but is not limited to SDS, triton, sodium hydroxide, or other similar lysing agents. The protease may be proteinase K or other proteases or mixtures of proteases. After lysis, proteins are precipitated using ammonium acetate, ammonium sulfate, protamine sulfate, or other methods for precipitating proteins. DNA is then precipitated using salt, such as sodium chloride, potassium chloride, ammonium chloride, or other salt and ethanol, or isopropanol, and redissolved. This preferred method for DNA extraction results in improved rates of successful amplification of liquid Pap samples as compared to other procedures. The DNA may also be extracted from fresh cells and from cells embedded in paraffin using methods that are known to those skilled in the art. An exemplary method is taught by Shibata et al. (Shibata, D. K. Arnheim, N. Martin, W. J., Detection of Human Papilloma Virus in Paraffin-Embedded Tissue using the Polymerase Chain Reaction, Journal of Experimental Medicine. 167(1)225-30, 1988) the disclosure of which is hereby incorporated by reference in its entirety. In a preferred method, DNA is first extracted from exfoliated cells collected in an alcohol based liquid fixative. Pap collection solutions and other solutions for clinical samples contain an alcohol based liquid fixative. The DNA extraction method from liquid based Pap collection solutions has been optimized for different forms of the Pap collection solutions, including the commercial products such as that sold under the trade name PreservCyt transport medium in the Thin Prep™ test by CyTyc Corporation and that sold under the trade name SurePath™ by TriPath Imaging.

In another aspect, the cell sample may be centrifuged and cells resuspended in a lysis buffer without the proteinase. A sample of the lysed cells is then used for PCR. Other methods for isolating DNA known by those skilled in the art may also be used.

Following extraction of DNA from the samples, the HPV sequence, if present in the sample, is amplified, either in a quantitative and/or qualitative manner. The amplification may be carried out using a variety of methods, including, but not limited to, polymerase chain reaction, ligase chain reaction, transcription-based amplification, DNA signal amplifications, such as branched DNA signal amplifications, Q-beta replication, boomerang DNA amplification, strand displacement activation, cycling probe technology, isothermal nucleic acid based amplification, or other self-sustained sequence replication assays.

Amplification of DNA from clinical samples by the polymerase chain reaction (PCR) is disclosed in U.S. Pat. Nos. 4,683,195 and 4,683,202, and the disclosures of which are incorporated herein by reference. In a preferred embodiment, DNA extracted from the cells from the clinical samples is amplified by PCR with consensus primers complimentary to known HPV sequences.

The consensus primers are a mixture of oligonucleotides. The consensus primers are typically a mixture of oligonucleotides of which at least a single pair is 100% complimentary, but not less than 95% complimentary, to known sequences of the genomes of HPV isolates. In one aspect, the consensus primers are designed to detect all known types of HPV.

Throughout the specification nucleotides are designated as follows.

| Symbol | Bases |
|---|---|
| G | G |
| A | A |
| T | T |
| C | C |
| R | G or A |
| Y | T or C |
| M | A or C |
| K | G or T |
| S | G or C |
| W | A or T |
| H | A, C or T |
| B | G, T or C |
| V | G, C or A |
| D | G, A or T |
| N | G, A, T, or C |

The novel oligonucleotide sequences include a 20 base pair forward primer (AGconF). Similarly, the novel oligonucleotide sequences include a reverse primer (AGconR). The sequences of the consensus primers, which amplify the L1 region of the HPV genome are:

Forward primer (AGconF):

(SEQ ID No:1)

5′-GCNCARGGHCAYAAYAATGG-3′
Reverse primer (AGconR):

(SEQ ID No:2)

5′-CGDCCHARRGGAWACTGATC-3′

The degeneracy of the consensus primers permits detection of a wide variety of HPV types, which contain sequence variations that cannot be detected by existing HPV consensus PCR primers. FIGS. 3 and 4 show the alignment of the forward and reverse consensus primers (AGconF and R), respectively, with sequences of a set of clinically important HPV types and compares them with the commonly used primers MYO9 and MYO11. Furthermore, FIG. 5 shows a direct comparison of the sequence diversity of the consensus primers compared to commonly used primers MY09 and MYO11, which is illustrated by the points of degeneracy with the primer mixtures for both systems. The figures highlight the points of consensus for these two primer pairs across the list of HPV types.

In a preferred embodiment, amplification with the two primers, AGconF and AGconR is carried out using PCR. An internal control is used to ensure that the amplification worked. An external control may also be used, but an internal control is preferred because an external control does not exclude the possibility that the sample was inadequate or carried an inhibitor of the particular gene chemistry.

Following amplification with primers AGconF and AGconR, the products are analyzed to detect the presence or absence of HPV DNA. This step may be performed using gel electrophoresis with agarose or polyacrylamide gels stained with ethidium bromide, CYBr green, or other dyes that emit fluorescence when stimulated by ultraviolet light. Alternatively, amplification products may be detected by the incorporation of a chemical fluorochrome attached to one of the nucleotide bases within the consensus primers, which is detected by direct fluorescence on gel or capillary electrophoresis. Other methods to separate DNA by size may also be used. Other methods, include but are not limited to, DNA amplification using oligonucleotide primers modified with additional bases on the 5′ end of the consensus sequence, where the added nucleotides are complementary to a DNA sequence fixed to the surface of a device or membrane to which the DNA product may hybridize. The hybridized PCR product may be visualized by a fluorescent DNA spot on a microarray or by an electronic signal, including a change in impedance, voltage or resistance. In another embodiment, amplified HPV sequences may be detected using biotin labeled probes attached to avidin on plates or microarrays. Alternatively, HPV sequences may be detected using peptide nucleic acid (PNA) probes. DNA Enzyme Immuno Assay (DEIA), Cleavase fragment length polymorphism (Third Wave, Madison, Wis.), dHPLC (denaturing high performance liquid chromatography), Massarray® SNP genotyping (Sequenom, San Diego, Calif.) or other DNA sequencing assays using MALDI-TOF mass spectroscopy analysis.

When analyzed by gel electrophoresis following amplification with primers AGconF and AGconR, the presence of HPV DNA is indicated by the presence of a 442-464 base pair band on the gels, representing the L1 region of HPV. Gel electrophoresis may be used to separate amplification reaction products. Negative samples, samples positive for HPV and samples weakly positive for HPV may be compared. The strength of the band intensity observed on a gel relates to viral load. The amplification reaction detects a broad range of HPV types, and has a very low sample failure rate. The reaction detects both circular and integrated viral genomes. An internal control in each sample may be included to indicate a successful amplification reaction.

The positive samples are next analyzed for the HPV genotype. Genotyping may be performed in several different ways. The preferred method is based on digestion of the PCR DNA products with a series of restriction endonucleases, resulting in a mixture of size-specific DNA fragments characteristic of each of the known HPV types. In one preferred embodiment of the invention, a series of three separate enzymes are used to fragment the DNA into patterns resolved by gel electrophoresis, where each separate enzyme reaction is analyzed in a separate lane of the gel. The restriction endonucleases cut the DNA product into a predictable series of DNA bands. The resulting pattern of bands is characteristic of each HPV type. One advantage of this method is the high degree of resolution for identifying the more than forty types typically observed in Pap samples. Additionally, in about 18% of cases, multiple HPV types are found. In these settings, combinations of multiple high risk HPVs, low and high-risk types, as well as, rare cases of low risk types only are seen. The sizes of the fragments following separation are indicative of high, intermediate or low risk HPVs. In another aspect, amplification products may be detected by genotype-specific probes, which include, but are not limited to, three or more oligonucleotide mixtures containing consensus sequences for HPV DNAs from high, medium and low risk HPVs.

Here, the amplification products may be blotted or applied to a membrane or other solid support, and probed with each of the labeled oligonucleotide mixtures. The label may be isotopic (³²P, ³⁵S, etc.) or non-isotopic (biotin, digoxigenin, etc.). Alternatively, amplification primers may be used that amplify only high, medium, or low risk HPV DNAs, or blocking primers may be used to block the amplification of low, medium and/or high risk HPV DNAs. The amplification products may be detected by gel electrophoresis, hybridization to an internal sequence or similar method.

“Molecular beacon” probes may also be used to determine the HPV genotype. These probes are described in Tyagi, et al., PCT application Nos. WO 95/13399 and WO 97/39008, the disclosures of which are hereby incorporated by reference. Molecular beacon probes contain a stem-and-loop structure in which the loop portion is complementary to the target nucleic acid sequence. The stem sequences are complementary to each other and labeled with fluorescent and quenching labels. When the loop portion hybridizes, the stem sequences are forced apart, which causes the molecule to fluoresce.

Computer software is used to aid in the detection and analysis of HPV DNA. After the initial gel electrophoresis to detect the presence or absence of HPV DNA, analyses of any samples proving negative for HPV DNA are completed by the generation of an interpretative report. Positive samples are re-selected from the detection work list by the computer software and a new HPV identification work list is generated for samples to be genotyped for HPV.

The present invention may also integrate interpretative database software known as PapFinder. The database uses restriction maps of the cataloged HPV types. Specifically, a catalog of the DNA bands created following digestion with a series of restriction endonucleases has been entered into a simple database to speed HPV typing. Each HPV type is cross-correlated with the assignment of its risk category and the relevant citations describing those viruses and their respective clinical behavior. Thus, the database can be used to genotype HPV species based on the use of the consensus primers of the present invention or for other primers commonly used for HPV detection.

When restriction endonuclease digestion is used to determine the HPV type, the completed gel may be imaged and the resulting file transmitted to an interpreter via the Internet with simultaneous transmission of the patient demographic information and other relevant clinical information.

The Pap-Finder software displays HPV fragment size results for the digestion of a particular HPV type with multiple or single restrictions enzymes. A preferred embodiment displays HPV fragment size obtained by digesting the L1 PCR fragment with PstI, RsaI, and HaeIII. The software also provides information about the particular HPV type and isolate. Web Portal version of the Pap-Finder software shows fragment sizes obtained by digesting the L1 PCR fragments of different HPV types with PstI, RsaI, HaeIII. The software also indicates the oncogenic risk associated with each HPV type. Searching of the database may be performed by HPV type, fragment sizes and other methods.

The present invention can be used not only to determine whether a clinical sample contains HPV, and also what type of HPV it contains. The invention may also be used to augment information about the frequency of HPV types, their distribution, and associated risk. Individual HPV types observed in clinical samples from multiple testing sites vary in frequency of observation between 0% and about 20%. Of the individual HPV types observed in clinical samples from multiple testing sites, about 13.26% are of low risk, about 7.72% are of intermediate risk, about 58.43% are of high risk and about 17.34% are of unknown risk. This invention has the potential to add to clinical data of the relative occurrence of individual HPV genotypes and their categorization into high, medium and low risk categories. The invention also has the potential for enabling the discovery of new HPV types and the risk associated with them.

### EXAMPLE 1

DNA is first extracted from exfoliated cells collected in an alcohol based liquid fixative. The liquid-cell suspension is centrifuged at 2000×g for 10 minutes to pellet the cells. The volume of the cell pellet is next determined by visual comparison to a template guide, which shows increasing sizes of pelleted cells and the corresponding volumes. The cell pellet is suspended in the determined volume of cell lysis solution, which contains EDTA, TRIS, SDS and proteinase K. The cell suspension is next agitated and heated. Protein is precipitated by adding ammonium acetate, mixing vigorously, and centrifuging for 10 minutes at 6000×g. The DNA is next precipitated by addition of isopropanol, and centrifugation for 10 minutes at 6000×g. The DNA pellet is washed with 70% ethanol, and the DNA solubilized in a TRIS-EDTA solution.

To amplify the DNA using PCR, an aliquot of the solubilized DNA extracted from cells in clinical samples is amplified using PCR. The amplification is carried out using a commercial thermocycler, including a ThermoHybaid Mx2 or a Eppendorf Mastercycler. A typical reaction mixture contains 20 pmol/μl of each consensus primer, 0.025 units/μl of Taq polymerase, 1×PCR buffer (as supplied with the commercial Taq polymerase), 4 mmoles/μl MgCl₂, 0.2 mM of each dNTP, and 50 μl of extracted DNA, and water to a total volume of 500 μl. When an amplification control is included in the reaction, instead of 20 pmol/μl of consensus primers, 13.3 pmol/l of each consensus primer and 1.67 pmol/μl of Beta-globin primers are added.

The sample of extracted DNA is added immediately before temperature cycling. The thermocycling parameters are: initial denaturation at 95° C. for 2 minutes, then for 40 cycles: denaturation at 95° C. for 20 seconds, annealing at 55° C. for 30 seconds, extension at 72° C. for 30 seconds, and after 40 cycles, a final extension at 72° C. for 5 minutes, and hold at 15° C. Appropriate controls are tested with each run, including a positive control containing DNA extracted from correlative sample type (ThinPrep or SurePath liquid collections) spiked with HPB positive cells from positive control cell lines. A negative control involves HPV negative control cell lines processed in the correlative sample type. A blank control involves all of the above listed reagents without added DNA and the missing volume replaced with water. Evaluation of the integrity of the PCR reaction is based on the detection of the DNA amplicon corresponding to the beta globin internal control. The sequences for the beta globin oligonucleotide primers are listed in Table Y.

Table Y:

Beta globin forward:
ACACAACTGTGTTCACCTAGC
Beta globin reverse:
GGAAAATAGACCAATAGGCAG

The PCR products are analyzed on 5% 1 mm precast polyacrylamide gels in TRIS-borate-EDTA buffer. 15 μl of the reaction mixture is loaded on the gels, and is electrophoresed at 120V for 50 minutes. The gel is next stained with ethidium bromide for 15-30 minutes.

PCR reactions indicating the presence of HPV DNA, indicated by a band of 442-464 bp are analyzed further to determine the HPV genotype. Aliquots of the PCR reactions are digested separately with the restriction enzymes Pst I, Rsa I and Hae III. The reactions contain 10 μl of the PCR reaction, 6.5 l of H₂O, 2 μl of 10× buffer (supplied with the restriction enzyme), 0.2 μl of 100× bovine serum albumin, and 1.3 ml of the restriction enzyme. The reactions are incubated at 37° C. for 2 hours, and electrophoresed on 5% TRIS-borate-EDTA polyacrylamide gels at 120V for 50 minutes. The gel is stained with ethidium bromide and imaged on a UPV series 8000 Digital Imaging System. The digital image is captured as a jpeg file.

The present invention may be assembled as a kit for detecting and typing HPV. The kit would contain solutions to extract DNA from clinical samples, template for determining the volume of the cell pellet, the consensus primers, and restriction enzymes to digest the PCR products Other kit components might include, but are not limited to the following: PCR buffers and enzymes, control primers, for example, beta-globin primers, and TBE polyacrylamide gels. The software may be utilized for visualization of distinct band sizes with various enzyme digests that can be compared with enzyme digests that are included in database for specific HPV types. Banding patterns in the database may allow the identification of HPV types present in a clinical sample by comparison with the bands sizes observed following gel electrophoresis or other size separation methods. Following comparison with these known band sizes, the relative frequency of occurrence of a clinical HPV isolate and its relative risk level can be assessed from the database.

The software in one specific example would display the band sizes for HPV type 58. The software would indicate that this HPV type is in supergroup A9, is a high risk type, and has an uncut L1 fragment size of 449 bp. Digestion of the L1 fragment with Pst I results in bands of 216, 207, and 26 bp. Digestion of the L1 fragment with Rsa I results in bands of 306, 111, and 32 bp. Digestion of the L1 fragment with Hae III results in an undigested band of 449 bp. A triple digest of the L1 fragment with Pst I, Rsa I, and Hae III results in band of 216, 111, 64, 32 and 25 bp. Corresponding results would be displayed for other HPV types.

What is claimed is:

1. An HPV probe selected from the group consisting of Sequence ID No: 1 and Sequence ID No: 2.

2. An HPV primer selected from the group consisting of Sequence ID No: 1 and Sequence ID No: 2.

3. A method for clinical analysis of Human Papilloma Virus, comprising:
removing a specimen from a patient;
isolating DNA from the specimen;
amplifying an L1 region in the DNA from the specimen using primers of Sequence ID No: 1 and Sequence ID No: 2 to produce an amplicon;
independently digesting the amplicon with a plurality of restriction enzymes;
determining the size of fragments from the digesting of the amplicon;
comparing fragment sizes from each independent digesting of the amplicon with restriction maps of digests of the amplicon from known types of human papilloma virus; and
categorizing the amplicon from the Human Papilloma virus into one of a high risk, a medium risk, a low risk and an indeterminate risk category.

4. A method, as in claim 1, further comprising determining the type of Human Papilloma Virus from the fragment sizes from the digest of the amplicon.


SEQUENCE LISTING (2 sequences)

SEQ ID NO: 1 - DNA, 20 bp, Human papillomavirus
  misc_feature (3)..(3): n is a, c, g, or t
gcncargghcayaayaatgg

SEQ ID NO: 2 - DNA, 20 bp, Human papillomavirus
cgdccharrggawactgatc